(19) United States
(12) Patent Application Publication
(10) Pub. No.: US 2014/0039071 A1
(43) Pub. Date: Feb. 6, 2014

(54) ISOPENTYL ESTERS FOR USE IN COSMETIC, DERMATOLOGICAL OR PHARMACEUTICAL COMPOSITIONS

(71) Applicants: Oliver Thum, Ratingen (DE); Marrit Friederike Eckstein, Essen (DE); Oliver Springer, Wesel (DE); Susann Wiechers, Essen (DE); Juergen Meyer, Essen (DE)

(72) Inventors: Oliver Thum, Ratingen (DE); Marrit Friederike Eckstein, Essen (DE); Oliver Springer, Wesel (DE); Susann Wiechers, Essen (DE); Juergen Meyer, Essen (DE)

(73) Assignee: EVONIK DEGUSSA GMBH, Essen (DE)

(21) Appl. No.: 14/006,811

(22) PCT Filed: Mar. 27, 2012
(86) PCT No.: PCT/EP12/55362

§ 371 Date: Sep. 23, 2013

(30) Foreign Application Priority Data
Mar. 29, 2011 (DE) 10 2011 006 362.5

(51) Int. Cl. A61K 8/37; A61Q 19/00

(52) U.S. Cl. CPC A61K 8/37; A61Q 19/00
USPC 514/785

(57) ABSTRACT

The invention provides the esters of a mixture of 2-methyl-1-butanol and 3-methyl-1-butanol and the use thereof for producing formulations.

### FIELD OF THE INVENTION

The invention relates to the esters of a mixture of 2-methyl-1-butanol and 3-methyl-1-butanol and to the use thereof for producing formulations.

### PRIOR ART

Cosmetic, dermatological or pharmaceutical formulations are manufactured in large amounts and consumed worldwide by consumers. Recently, requirements have increased as regards the sustainability of the formulations and their ingredients. In particular, there is a need to produce as many as possible of the components used on the basis of renewable raw materials.

At the same time, however, consumer requirements are also increasing as regards the sensory properties of the formulations to be used, especially the haptic properties (e.g. oiliness, waxiness) and olfactory properties. This is especially true for formulations which have a high fraction of active ingredients in dispersed form (for example inorganic UV filters such as TiO₂ or ZnO) or dissolved form (e.g. organic UV filters or urea).

EP2243517A1 describes the use of esters of isoamyl alcohol (3-methyl-1-butanol), especially of lauric acid esters of 3-methyl-1-butanol, in selected sunscreen formulations, i.e. in cosmetic formulations comprising inorganic UV filters and/or organic UV filters from the group of so-called “triazines”. Here, it is described how such sunscreen formulations have an improved skin feel compared to formulations based on established components, e.g. based on butylene glycol esters or C12-C15 alkyl benzoate.

However, EP2243517A1 does not describe by way of example which specific sensory properties are improved through the use of the esters according to the invention. Furthermore, only sunscreen formulations are expressly mentioned.

“Emollients in Personal Care for Solubilizing Organic UV Absorbers” (IP.COM Journal, IP.COM Inc., West Henrietta, N.Y., US, 22 February 2006, XP013112931, ISSN 1533-0001) likewise discloses a number of components which are suitable for producing sunscreen formulations with good spreadability on skin and good absorption capacity into skin, for example isoamyl laurate (cf. p. 8).

Here too only sunscreen formulations are expressly mentioned.

There is also a need for components for cosmetic, dermatological or pharmaceutical formulations which bring about improved sensory properties of precisely these formulations.

It was therefore an object of the present invention to develop new types of cosmetic ingredients which permit production of cosmetic, dermatological or pharmaceutical formulations with improved sensory properties.

### DESCRIPTION OF THE INVENTION

Surprisingly, it has been found that esters based on a mixture of 3-methyl-1-butanol and 2-methyl-1-butanol meet these requirements. Such an isomer mixture of isopentyl alcohol can be obtained from the residues of alcoholic fermentation and therefore constitutes a natural raw material.

Compared to formulations based on known ingredients, e.g. esters of pure 3-methyl-1-butanol, the formulations according to the invention exhibit less of an oily skin feel, less stickiness, and an improved odour.

The present invention therefore provides ester mixtures of at least one optionally branched, optionally unsaturated, optionally substituted carboxylic acid with a chain length of 6 to 30, in particular 8 to 22, preferably 10 to 16, carbon atoms with 2-methyl-1-butanol and 3-methyl-1-butanol, where the weight ratio of 2-methyl-1-butanol and 3-methyl-1-butanol is from 0.05:1 to 1:0.05, in particular from 0.1:1 to 1:1, preferably from 0.1:1 to 0.67:1.

The acid components preferably used for producing the esters according to the invention are optionally branched, optionally unsaturated, optionally substituted acids having 6 to 30 carbon atoms, in particular with 8 to 22 carbon atoms, such as caproic acid, caprylic acid, capric acid, lauric acid, myristic acid, palmitic acid, palmitoleic acid, isostearic acid, stearic acid, 12-hydroxystearic acid, dihydroxystearic acid, oleic acid, linoleic acid, petroselic acid, elaidic acid, arachic acid, behenic acid, erucic acid, gadoleic acid, linolenic acid, eicosapentaenoic acid, docosahexaenoic acid, arachidonic acid, which can be used on their own or in a mixture, in particular lauric acid, myristic acid, palmitic acid and stearic acid, mixtures of coconut fatty acids, in particular completely hydrogenated coconut fatty acids, particularly preferably completely hydrogenated coconut fatty acids with a content of fatty acids of chain lengths C12 to C18 of together >98% by weight, based on the sum of all carboxylic acids.

Here, particular preference is given to using monobasic, optionally unsaturated fatty acids having 12-18 carbon atoms, such as lauric acid, myristic acid, palmitic acid, palmitoleic acid, isostearic acid, stearic acid, oleic acid, linoleic acid, linolenic acid, which can be used alone or in a mixture.

Particular preference is given here to lauric acid, myristic acid, palmitic acid and stearic acid, which can be used alone or in a mixture.

In a further embodiment, dibasic acids with 4 to 20 carbon atoms, for example adipic acid or sebacic acid, are used as acid component.

The alcohol mixture used is a mixture of 2-methyl-1-butanol and 3-methyl-1-butanol with a weight ratio of 2-methyl-1-butanol to 3-methyl-1-butanol of 0.05:1 to 1:0.05, in particular from 0.1:1 to 1:1, preferably from 0.1:1 to 0.67:1.

The mixing ratio of the alcohols used can be determined both for this mixture and also for the resulting esterification products by means of 1H-NMR spectroscopy using standard methods and standard solvents (e.g. CDCl₃ or DMSO-d6).

The esters can be produced by the methods known to the person skilled in the art, as described for example in ORGANIKUM (Wiley-VCH, 22nd Edition 2004, pages 474-480). Typical processes for producing cosmetic esters utilize acids, e.g. p-TSA, or metal salts (e.g. tin or titanium salts) or enzymes (for example lipases), as catalyst, see e.g. EP1816154A1.

The ester mixtures according to the invention can be obtained directly either as described in the examples by esterification of the mixture of 2-methyl-1-butanol and 3-methyl-1-butanol, although it is likewise possible in accordance with the invention to synthesize the esters of 2-methyl-1-butanol and 3-methyl-1-butanol separately and then to mix them accordingly, although preference is given according to the invention to the first-mentioned one-pot reaction.

The present invention further provides cosmetic, dermatological or pharmaceutical formulations comprising an ester mixture according to the invention.

The formulations according to the invention can be produced as water-in-oil, oil-in-water or water-in-silicone emulsions.

The invention further provides the use of the ester mixtures according to the invention for producing optionally dispersed-solids-containing cosmetic, dermatological or pharmaceutical formulations or care and cleaning compositions for the home or industry, in particular for hard surfaces, leather or textiles. Consequently, the cosmetic, dermatological or pharmaceutical formulations, care and cleaning compositions for the home or industry and the care and cleaning compositions for hard surfaces, leather or textiles comprising an ester mixture according to the invention are likewise provided by the present invention.

The formulation according to the invention can comprise for example at least one additional component selected from the group of

emollients,

emulsifiers,

thickeners/viscosity regulators/stabilizers,

antioxidants,

hydrotropes (or polyols),

solids and fillers,

pearlescent additives,

deodorant and antiperspirant active ingredients,

insect repellents,

self-tanning agents,

preservatives,

conditioners,

perfumes,

dyes,

cosmetic active ingredients,

care additives,

superfatting agents,

solvents.

Substances which can be used as exemplary representatives of the individual groups are known to the person skilled in the art and can be found for example in EP2273966A1. This patent application is hereby incorporated by reference and thus forms part of the disclosure.

As regards further optional components and the amounts of these components used, reference is made expressly to the relevant handbooks known to the person skilled in the art, for example K. Schrader, “Grundlagen and Rezepturen der Kosmetika [Fundamentals and formulations of cosmetics]”, 2nd Edition, pages 329 to 341, Hüthig Buch Verlag Heidelberg.

The amounts of the respective additives are governed by the intended use.

Typical guide formulations for the respective applications are known prior art and are contained for example in brochures from the manufacturers of the particular basic materials and active ingredients. These existing formulations can generally be adopted unchanged. If required, the desired modifications can, however, be undertaken without complication by simple experiments for the purposes of adaptation and optimization.

The present invention is described by way of example in the examples listed below without any intention of limiting the invention, the scope of application of which arises from the entire description and the claims, to the embodiments specified in the examples.

Unless stated otherwise, all of the stated percentages (%) are percentages by mass.

### EXAMPLES

### Example 1

### Preparation of 2-methyl-1-butyl laurate (Comparative Example)

160 g of 2-methyl-1-butanol and 300 g of lauric acid are charged to a multi-neck round-bottomed flask and heated to 60° C. After adding 1.5 g of Novozym 435, a vacuum is applied (ca. 120 mbar) until the reaction mixture boils. The water of reaction which is formed is separated off with the help of a water separator and the distilled-off alcohol is returned. After 10 hours, the immobilized enzyme is filtered off and the excess alcohol is removed by distillation. By means of subsequent steam distillation (2 hours, 120° C.), the residual content of 2-methyl-1-butanol is reduced to 20 ppm. The product is dried (1 hour, 120° C., 20 mbar) and is produced as a colourless liquid without further work-up.

### Example 2

### Preparation of 3-methyl-1-butyl laurate (Comparative Example)

160 g of 3-methyl-1-butanol and 300 g of lauric acid are charged to a multi-neck round-bottomed flask and heated to 60° C. After adding 1.5 g of Novozym 435, a vacuum is applied (ca. 120 mbar) until the reaction mixture boils. The water of reaction that is formed is separated off with the help of a water separator and the distilled-off alcohol is returned. After 10 hours, the immobilized enzyme is filtered off and the excess alcohol is removed by distillation. As a result of subsequent steam distillation (2 hours, 120° C.), the residual content of 3-methyl-1-butanol is reduced to 20 ppm. The product is dried (1 hour, 120° C., 20 mbar) and is formed as a colourless liquid without further work-up.

### Example 3

### Preparation of a Mixture According to the Invention of 2-methyl-1-butyl laurate and 3-methyl-1-butyl laurate

64 g of 2-methyl-1-butanol, 96 g of 3-methyl-1-butanol and 300 g of lauric acid are charged to a multi-neck round-bottomed flask and heated to 60° C. After adding 1.5 g of Novozym 435, a vacuum is applied (ca. 120 mbar) until the reaction mixture boils. The water of reaction that is formed is separated off with the help of a water separator and the distilled-off alcohol is returned. After 10 hours, the immobilized enzyme is filtered off and the excess alcohol is removed by distillation. As a result of subsequent steam distillation (2 hours, 120° C.), the residual content of 2-methyl-1-butanol and 3-methyl-1-butanol is reduced to together 20 ppm. The product is dried (1 hour, 120° C., 20 mbar) and is formed as a colourless liquid without further work-up.

### Example 4

### Preparation of 2-methyl-1-butylcocoate (Comparative Example)

150 g of 2-methyl-1-butanol and 300 g of coconut fatty acid (completely hydrogenated, distilled coconut fatty acid with a content of fatty acids of chain lengths C12 to C18 of together >98%) are charged to a multi-neck round-bottomed flask and heated to 60° C. After adding 1.5 g of Novozym 435, a vacuum is applied (ca. 120 mbar) until the reaction mixture boils. The water of reaction that is formed is separated off with the help of a water separator and the distilled-off alcohol is returned. After 10 hours, the immobilized enzyme is filtered off and the excess alcohol is removed by distillation. As a result of subsequent steam distillation (2 hours, 120° C.), the residual content of 2-methyl-1-butanol is reduced to 20 ppm. The product is dried (1 hour, 120° C., 20 mbar) and is formed as a colourless liquid without further work-up.

### Example 5

### Preparation of 3-methyl-1-butylcocoate (Comparative Example)

150 g of 3-methyl-1-butanol and 300 g of coconut fatty acid (completely hydrogenated, distilled coconut fatty acid with a content of fatty acids of chain lengths C12 to C18 of together >98%) are charged to a multi-neck round-bottomed flask and heated to 60° C. After adding 1.5 g of Novozym 435, a vacuum is applied (ca. 120 mbar) until the reaction mixture boils. The water of reaction that is formed is separated off with the help of a water separator and the distilled-off alcohol is returned. After 10 hours, the immobilized enzyme is filtered off and the excess alcohol is removed by distillation. As a result of subsequent steam distillation (2 hours, 120° C.), the residual content of 3-methyl-1-butanol is reduced to 20 ppm. The product is dried (1 hour, 120° C., 20 mbar) and is formed as a colourless liquid without further work-up.

### Example 6

### Preparation of a Mixture According to the Invention of 2-methyl-1-butyl-cocoate and 3-methyl-1-butylcocoate

64 g of 2-methyl-1-butanol, 96 g of 3-methyl-1-butanol and 300 g of coconut fatty acid (completely hydrogenated, distilled coconut fatty acid with a content of fatty acids of chain lengths C12 to C18 of together >98%) are charged to a multineck round-bottomed flask and heated to 60° C. After adding 1.5 g of Novozym 435, a vacuum is applied (ca. 120 mbar) until the reaction mixture boils. The water of reaction that is formed is separated off with the help of a water separator and the distilled-off alcohol is returned. After 10 hours, the immobilized enzyme is filtered off and the excess alcohol is removed by distillation. As a result of subsequent steam distillation (2 hours, 120° C.), the residual content of 2-methyl-1-butanol and 3-methyl-1-butanol is reduced to together 20 ppm. The product is dried (1 hour, 120° C., 20 mbar) and is formed as a colourless liquid without further work-up.

### Example 7

### Preparation of a Mixture According to the Invention of 2-methyl-1-butylcocoate and 3-methyl-1-butylcocoate

125 g of 3-methyl-1-butanol, 25 g of 2-methyl-1-butanol and 300 g of coconut fatty acid (completely hydrogenated, distilled coconut fatty acid with a content of fatty acids of chain lengths C12 to C18 of together >98%) are charged to a multi-neck round-bottomed flask and heated to 60° C. After adding 1.5 g of Novozym 435, a vacuum is applied (ca. 120 mbar) until the reaction mixture boils. The water of reaction that is formed is separated off with the help of a water separator and the distilled-off alcohol is returned. After 10 hours, the immobilized enzyme is filtered off and the excess alcohol is removed by distillation. As a result of subsequent steam distillation (2 hours, 120° C.), the residual content of 2-methyl-1-butanol and 3-methyl-1-butanol is reduced to together 20 ppm. The product is dried (1 hour, 120° C., 20 mbar) and is formed as a colourless liquid without further work-up.

### Example 8

### Application Examples

All concentrations in the application examples are given in percentages by weight. To produce the emulsions, customary homogenization processes used for oil-in-water and water-in-oil emulsions and known to the person skilled in the art are used.

### Difference in the Performance Compared with the Prior Art

These experiments aim to show that the mixed esters according to the invention of 2-methyl-1-butanol and 3-methyl-1-butanol and lauric acid bring about advantages with regard to sensory properties and the product odour compared to the respective pure esters.

### Example 8.1

### Testing of Laurate Esters in O/W Lotions

The test formulations V1, V2 and 1 are O/W lotions. In this connection, oil phase A and water phase B were each heated to 75° C., combined and then homogenized using a suitable homogenizer (e.g. Ultraturrax) for ca. 1-2 minutes.

Stabilizing polymers (xanthan gum, carbomer) were stirred into the emulsion as an oily dispersion at temperatures of 50-60° C. Homogenization was carried out for one minute.

The addition of further ingredients (e.g. preservatives, active ingredients) preferably takes place at 40° C.

In the formulation examples, the individual raw materials are listed with their INCI name.

TABLE 1
O/W lotion test formulations

| | | V1 | V2 | 1 |
|---|---|---|---|---|
| A | Polyglyceryl-3 Dicitrate/Stearate¹⁾ | 2.5 | 2.5 | 2.5 |
| | Ester from Example 1 | 15.0 | — | — |
| | Ester from Example 2 | — | 15.0 | — |
| | Ester from Example 3 | — | — | 15.0 |
| B | Glycerin | 3.0 | 3.0 | 3.0 |
| | Demineralized Water | ad 100 | ad 100 | ad 100 |
| C | Carbomer | 0.1 | 0.1 | 0.1 |
| | Xanthan Gum | 0.3 | 0.3 | 0.3 |
| | Ethylhexyl Palmitate | 0.8 | 0.8 | 0.8 |
| D | NaOH 10% aq | q.s. | q.s. | q.s. |
| E | Phenoxyethanol, Ethylhexylglycerin²⁾ | 0.8 | 0.8 | 0.8 |

¹⁾TEGO ® Care PSC 3 (Evonik Goldschmidt GmbH)
²⁾Euxyl ® PE 9010 (Schülke)

Test formulations 1, V1 and V2 were investigated by a trained sensory subject group (N=13). The skin feel of the cosmetic formulations described in the examples was determined by a so-called panel. For this, a defined amount of the test lotions were applied to a clearly defined area of the forearm. The subjects compared the sensory properties of the cosmetic formulations and of the respective comparison formulation without knowing the composition. Assessment was made on a scale from 0 (little) to 10 (a lot).

Whereas no differences as regards application properties such as “spreadability” and “lubricity” could be found relative to the formulations comprising the comparison emollients, there were noticeable differences as regards the properties “Absorption”, “oiliness” and “stickiness”.

Moreover, the group assessed the odour of the formulations on a scale of from 1 (good) to 3 (poor).

Table 2 summarizes the average values of the described tests.

TABLE 2
Sensory and olfactory comparison in O/W test emulsions

| Formu- | Spread- | Ab- | | | | |
|---|---|---|---|---|---|---|
| lation | ability | sorption | Oiliness | Stickiness | Lubricity | Odour |
| V1 | 8 | 4 | 4 | 4 | 5 | 1.4 |
| V2 | 7 | 5 | 4 | 3 | 5 | 1.5 |
| 1 | 8 | 7 | 2 | 1 | 4 | 1.2 |

### Example 8.2

### Testing of Cocoate Esters in W/O Creams

Test formulations V3, V4 and 2 are W/O creams. To produce them, the oil phase A was heated to 80° C. The water phase was then added slowly to the oil phase with stirring and combined and then homogenization was carried out using a suitable homogenizer (e.g. Ultraturrax) for ca. 1-2 minutes.

After adding the preservative (below 40° C.), homogenization is again briefly carried out below 30° C.

TABLE 3
W/O cream test formulations

| | | V3 | V4 | 2 |
|---|---|---|---|---|
| A | Diisostearoyl Polyglyceryl-3 | 3.0 | 3.0 | 3.0 |
| | Dimer Dilinoleate³⁾ | | | |
| | Ester from Example 4 | 19.0 | — | — |
| | Ester from Example 5 | — | 19.0 | — |
| | Ester from Example 6 | — | — | 19.0 |
| | Hydrogenated Castor Oil | 0.5 | 0.5 | 0.5 |
| | Beeswax | 0.5 | 0.5 | 0.5 |
| B | Glycerin | 3.0 | 3.0 | 3.0 |
| | Demineralized Water | ad 100 | ad 100 | ad 100 |
| | Magnesium Sulfate Heptahydrate | 1.0 | 1.0 | 1.0 |
| D | NaOH 10% aq | q.s. | q.s. | q.s. |
| E | Sodium Benzoate, Potassium | 0.8 | 0.8 | 0.8 |
| | Benzoate, Aqua⁴⁾ | | | |

³⁾ISOLAN ® PDI (Evonik Goldschmidt GmbH)
⁴⁾Euxyl ® K 712 (Schülke)

Table 4 summarizes the assessments of the sensory and olfactory panel for the W/O creams.

TABLE 4
Sensory and olfactory comparison in W/O test formulations

| Formu- | Spread- | Ab- | | | | |
|---|---|---|---|---|---|---|
| lation | ability | sorption | Oiliness | Stickiness | Lubricity | Odour |
| V3 | 4 | 3 | 6 | 5 | 2 | 1.7 |
| V4 | 4 | 3 | 7 | 5 | 2 | 1.8 |
| 2 | 4 | 5 | 4 | 3 | 3 | 1.4 |

The comparison in both test emulsions consistently shows that the mixed esters according to the invention have sensory and olfactory advantages compared with the pure esters in the prior art.

### Examples of the Use of the Esters According to the Invention in Cosmetic Formulations

### Example 9

### Natural O/W Body Lotion

| | | |
|---|---|---|
| A | Polyglyceryl-3 Dicitrate/Stearate¹⁾ | 2.50% |
| | Ester from Example 7 | 7.00% |
| | Almond Oil | 5.00% |
| | Caprylic/Capric Triglyceride²⁾ | 5.50% |
| B | Water | 75.50% |
| | Glycerin | 3.00% |
| C | Xanthan Gum | 0.50% |
| D | Sodium Hydroxide (10%) | q.a. |
| E | Benzyl Alcohol; Glycerin; Benzoic Acid; Sorbic Acid³⁾ | 0.80% |

¹⁾TEGO ® Care PSC 3 (Evonik Goldschmidt GmbH)
²⁾TEGOSOFT ® CT (Evonik Goldschmidt GmbH)
³⁾Rokonsal ® BSB-N (ISP)

### Example 10

### Natural W/O Lotion

| | | |
|---|---|---|
| A | Polyglyceryl-4 Diisostearate/Polyhydroxystearate/Sebacate¹⁾ | 2.50% |
| | Caprylic/Capric Triglyceride²⁾ | 7.50% |
| | Ester from Example 7 | 6.00% |
| | Jojoba Oil | 5.00% |
| | Hyd. Castor Oil | 0.40% |
| | Beeswax | 0.60% |
| B | Water | 73.00% |
| | Glycerin | 3.00% |
| | Panthenol | 0.50% |
| | Mg SO₄ × 7H₂O | 1.00% |
| | Sodium Benzoate, Potassium Benzoate, Aqua³⁾ | 0.50% |

ISOLAN ® GPS (Evonik Goldschmidt GmbH)
TEGOSOFT ® CT (Evonik Goldschmidt GmbH)
Euxyl ® K712 (Schülke & Mayr)

### Example 11

### Natural O/W Cream

| | | |
|---|---|---|
| A | Polyglyceryl-3 Distearate/Stearate¹⁾ | 3.00% |
| | Glyceryl Stearate²⁾ | 2.00% |
| | Stearyl Alcohol³⁾ | 1.00% |
| | Decyl Cocoate⁴⁾ | 2.90% |
| | Ester from Example 7 | 5.00% |
| | Oleyl Erucate⁵⁾ | 2.00% |
| | Almond Oil | 10.00% |
| | Ceramide III | 0.10% |
| | Water | 70.20% |
| B | Glycerin | 3.00% |
| | Benzyl Alcohol; Glycerol, Benzoic Acid; Sorbic Acid⁶⁾ | 0.80% |
| D | Sodium Hydroxide (10%) | q.a. |

¹⁾TEGO ® Care PSC 3 (Evonik Goldschmidt GmbH)
²⁾TEGIN ® M (Evonik Goldschmidt GmbH)
³⁾TEGO ® Alkanol 18 (Evonik Goldschmidt GmbH)
⁴⁾TEGOSOFT ® DC (Evonik Goldschmidt GmbH)
⁵⁾TEGOSOFT ® OER (Evonik Goldschmidt GmbH)
⁶⁾Rokonsal ® BSB-N (ISP)

### Example 12

### O/W Serum

| | | |
|---|---|---|
| A | Glyceryl Stearate; PEG-100 Stearate¹⁾ | 3.00% |
| | Stearyl Alcohol²⁾ | 0.50% |
| | Caprylic/Capric Triglyceride³⁾ | 5.00% |
| | Cetyl Ricinoleate⁴⁾ | 1.00% |
| | Ester from Example 7 | 5.50% |
| B | Water | 76.125% |
| | Glycerin | 3.00% |
| | Tetrapeptide-21; Glycerin; Butylene Glycol; Aqua⁵⁾ | 2.50% |
| | Glycerin, Tetrapeptide-30⁶⁾ | 2.50% |
| | Carbomer⁷⁾ | 0.0075% |
| C | Sodium Hydroxide (10%) | q.a. |
| Z | Dipropylene Glycol; Methyl Paraben; Ethyl Paraben; | 0.80% |
| | Aqua; Methylisothiazolinone⁸⁾ | |

TEGO ® Care 165 (Evonik Goldschmidt GmbH)
TEGO ® Alkanol 18 (Evonik Goldschmidt GmbH)
TEGOSOFT ® CT (Evonik Goldschmidt GmbH)
TEGOSOFT ® CR (Evonik Goldschmidt GmbH)
TECO ® PEP 4-17 (Evonik Goldschmidt GmbH)
TEGO ® Pep 4 Even (Evonik Goldschmidt GmbH)
TEGO ® Carbomer 141 G (Evonik Goldschmidt GmbH)
Microcare ® MEM (Thor GmbH)

### Example 13

### O/W Sunscreen Lotion SPF 30

| | | |
|---|---|---|
| A | Glyceryl Stearate Citrate¹⁾ | 2.50% |
| | Cetearyl Alcohol²⁾ | 1.00% |
| | Caprylic/Capric Triglyceride³⁾ | 3.00% |
| | Isopropyl Myristate⁴⁾ | 3.00% |
| | Ester from Example 7 | 5.00% |
| | Bis-Ethylhexyloxyphenol Methoxyphenyl Triazine⁵⁾ | 3.00% |
| | Diethylamino Hydroxybenzoyl Hexyl Benzoate⁶⁾ | 2.50% |
| | Octocrylene | 4.00% |
| | Ethylhexyl Salicylate | 4.00% |
| | Ethylhexyl Triazone | 1.50% |
| B | Titanium Dioxide; Trimethoxycaprylylsilane⁷⁾ | 3.00% |
| | Xanthan Gum | 0.20% |
| | Carbomer⁸⁾ | 0.20% |
| C | Glycerin | 3.00% |
| | Water | 62.70% |
| D | Sodium Hydroxide (10%) | q.a. |
| Z | Dipropylene Glycol; Methyl Paraben; Ethyl Paraben; | 0.80% |
| | Aqua; Methylisothiazolinone⁹⁾ | |
| | Perfume | q.a. |

¹⁾AXOL ® C 62 (Evonik Goldschmidt GmbH)
²⁾TEGO ® Alkanol 1618 (Evonik Goldschmidt GmbH)
³⁾TEGOSOFT ® CT (Evonik Goldschmidt GmbH)
⁴⁾TEGOSOFT ® M (Evonik Goldschmidt GmbH)
⁵⁾Tinosorb ® S (BASF SE)
⁶⁾Uvinul ® A Plus (BASF SE)
⁷⁾TEGO ® Sun T 805 (Evonik Goldschmidt GmbH)
⁸⁾TEGO ® Carbomer 141 G (Evonik Goldschmidt GmbH)
⁹⁾Microcare ® MEM (Thor GmbH)

### Example 14

### W/O Emulsion for an Antiperspirant Aerosol

| | | |
|---|---|---|
| A | Cetyl PEG/PPG-10/1 Dimethicone¹⁾ | 2.00% |
| | Polyglyceryl-4 Diisostearate/Polyhydroxystearate/Sebacate²⁾ | 2.00% |
| | Ester from Example 7 | 25.00% |
| | C₂₋₁₅ Alkyl Benzoate³⁾ | 8.00% |
| | Perfume | 3.00% |
| B | Water | 34.20% |
| | Aluminium Chlorohydrate | 20.00% |
| | Propylene Glycol | 5.00% |
| C | Phenoxyethanol; Ethylhexylglycerol⁴⁾ | 0.80% |

¹⁾ABIL ® EM 90 (Evonik Goldschmidt GmbH)
²⁾ISOLAN ® GPS (Evonik Goldschmidt GmbH)
³⁾TEGOSOFT ® TN (Evonik Goldschmidt GmbH)
⁴⁾Euxyl ® PE 9010 (Schülke & Mayr)

### Example 15

### Anhydrous Concentrate for Antiperspirant Aerosol

| | | |
|---|---|---|
| A | Ester from Example 7 | 47.00% |
| | Disteardimonium Hectorite | 3.00% |
| | Diethylhexyl Carbonate¹⁾ | 10.00% |
| | Silica Dimethyl Silylate²⁾ | 2.00% |
| | Perfume | 5.00% |
| B | C₂₋₁₅ Alkyl Benzoate³⁾ | 8.00% |
| | Aluminium Chlorohydrate | 25.00% |

TEGOSOFT ® DEC (Evonik Goldschmidt GmbH)
AEROSIL ® R 972 (Evonik Degussa GmbH)
TEGOSOFT ® TN (Evonik Goldschmidt GmbH)

The anhydrous concentrate from Example 8 can be combined with customary propellant gas mixtures such as e.g. propane/butane/isopropane/isobutane in the ratio of 25 to 50 parts of emulsion to 75 to 50 parts of propellant gas as aerosol.

### Example 16

### W/O Make-Up Formulation

| | | |
|---|---|---|
| A | Polyglyceryl-4 Isostearate; Cetyl PEG/PPG-10/1 | 6.0% |
| | Dimethicone; Hexyl Laurate¹⁾ | |
| | Cetyl Dimethicone | 1.0% |
| | Ester from Example 7 | 4.5% |
| | Ethylhexyl Palmitate | 1.5% |
| | Dimethicone | 5.0% |
| | Cyclopentasiloxane | 9.0% |
| | Phenyl Trimethicone | 1.0% |
| | Lauryl Dimethicone/Polyglyceryl-3 Crosspolymer; | 2.0% |
| | Triethylhexanoin²⁾ | |
| | Nylon-12 | 1.0% |
| | Iron Oxides | 2.0% |
| | Titanium Oxide | 6.0% |
| | Zinc Oxide | 0.5% |
| B | Glycerin | 3.0% |
| | Sodium Chloride | 0.8% |
| | Creatine | 0.2% |
| | Water | ad 100% |
| Z | Preservative, Perfume | q.s. |

¹⁾ABIL ® WE 09 (Evonik Goldschmidt GmbH)
²⁾KSG-830 (Shin-Etsu)

1. An ester mixture of at least one carboxylic acid with a chain length of 6 to 30 carbon atoms, with 2-methyl-1-butanol and 3-methyl-1-butanol, where the weight ratio of 2-methyl-1-butanol and 3-methyl-1-butanol is from 0.05:1 to 1:0.05.

2. The ester mixture of claim 1, wherein the carboxylic acid is selected from the group consisting of caproic acid, caprylic acid, capric acid, lauric acid, myristic acid, palmitic acid, palmitoleic acid, isostearic acid, stearic acid, 12-hydroxystearic acid, dihydroxystearic acid, oleic acid, linoleic acid, petroselic acid, elaidic acid, arachic acid, behenic acid, erucic acid, gadoleic acid, linolenic acid, eicosapentaenoic acid, docosahexaenoic acid, arachidonic acid, and mixtures of coconut fatty acids.

3. (canceled)

4. A formulation comprising an ester mixture of at least one carboxylic acid with a chain length of 6 to 30 carbon atoms, with 2-methyl-1-butanol and 3-methyl-1-butanol, where the weight ratio of 2-methyl-1-butanol and 3-methyl-1-butanol is from 0.05:1 to 1:0.05, and at least additional component.

5. The ester mixture of claim 1, wherein said chain length of said carboxylic acid is from 8 to 22 carbon atoms.

6. The ester mixture of claim 1, wherein said chain length of said carboxylic acid is from 10 to 16 carbon atoms.

7. The ester mixture of claim 1, wherein said weight ratio of 2-methyl-1-butanol and 3-methyl-1-butanol is from 0.1:1 to 1:1.

8. The ester mixture of claim 1, wherein said weight ratio of 2-methyl-1-butanol and 3-methyl-1-butanol is from 0.1:1 to 0.67:1.

9. The ester mixture of claim 2, wherein said carboxylic acid is selected from the group consisting of lauric acid, myristic acid, palmitic acid and stearic acid.

10. The ester mixture of claim 2 wherein said carboxylic acid comprises a mixture of completely hydrogenated coconut fatty acids.

11. The ester mixture of claim 2 wherein said carboxylic acid comprises a mixture of completely hydrogenated coconut fatty acids with a content of fatty acids of chain lengths C12 to C18 of together >98% by weight, based on the sum of all carboxylic acids.

12. The ester mixture of claim 1, wherein said carboxylic ester is a branched carboxylic acid.

13. The ester mixture of claim 1, wherein said carboxylic ester is an unsaturated carboxylic acid.

14. The ester mixture of claim 1, wherein said carboxylic ester is a substituted carboxylic acid.
